(19) United States
(12) Patent Application Publication
(10) Pub. No.: US 2012/0270922 A1
(43) Pub. Date: Oct. 25, 2012

(54) Antiangiogenic Agent and Method for Inhibition of Angiogenesis

(76) Inventors: Nobuyuki Takakura, Suita-shi (JP); Hiroyasu Kidoya, Suita-shi (JP); Fumitaka Muramatsu, Suita-shi (JP)

(21) Appl. No.: 13/392,196

(22) PCT Filed: Aug. 26, 2010
(86) PCT No.: PCT/JP2010/064523

§ 371 Date: Feb. 24, 2012

(30) Foreign Application Priority Data
Aug. 26, 2009 (JP) 2009 195848

(51) Int. Cl. A61K 31/7052; A61P 27/02; A61P 29/00; A61P 35/00; A61P 9/00

(52) U.S. Cl. 514/44 A

(57) ABSTRACT

This invention provides an antiangiogenic agent having a higher treatment effect than those of conventional antiangiogenic agents, and a method for inhibiting angiogenesis using the same. An antiangiogenic agent comprising at least one miRNA type selected from the group consisting of miRNAs, pre-miRNAs, and pri-miRNAs, each having a miRNA activity on VE-cadherin.

### TECHNICAL FIELD

The present invention relates to an antiangiogenic agent, and a method for inhibiting angiogenesis.

### BACKGROUND ART

Angiogenesis is considered to involve various diseases. It has been reported that angiogenesis involves formation of tumor tissues, formation of diseased tissues in chronic rheumatoid arthritis and other chronic inflammatory diseases, formation of excessive angiogenesis, which is the main cause of diabetic retinopathy, and the like. Recently, isolation of factors involving angiogenesis (hereinafter sometimes referred to as “angiogenesis factors”) and the function analysis thereof have been advanced.

Using these findings, antiangiogenic agents to improve various diseases have been developed. Many of these antiangiogenic agents have an effect of inhibiting the activity of angiogenesis factors. So far, clinical examples that Avastin (registered trademark), which is a neutralizing antibody of vascular endothelial growth factor (VEGF), shows a certain effect, increases the average life expectancy of terminal colon cancer patients, and is effective for diabetic retinopathy, have been reported (Non-patent Literature 1).

However, conventional antiangiogenic agents and methods for inhibiting angiogenesis using these agents do not attain sufficient treatment effects. In particular, although the conventional antiangiogenic agents can destroy blood vessels in the central region of a tumor, most of the blood vessels present around the tumor are mature (Non-patent Literature 2), and the conventional antiangiogenic agents cannot destroy these mature blood vessels. There have been many reports regarding cancer recurrence (Non-patent Literature 3) and cancer invasion/metastasis (Non-patent Literature 4 and 5) from the surrounding regions of the tumor after the administration of an antiangiogenesis agent. It is believed that cancer cells remaining around mature blood vessels, which are present around the tumor and have resistance after the administration of an antiangiogenic agent, are causes of recurrence, invasion, and metastasis. It has been revealed that a cancer stem cell, which is the highest grade cancer in cancer cells and is considered a cause of cancer recurrence or metastasis, is increased in a blood vessel region as an ecologically appropriate place. In particular, cancer stem cells are concentrated on the blood vessel region around the tumor (Non-patent Literature 6). In recent years, it has been indicated that a VEGF therapeutic agent, etc., leads to maturation of blood vessels (Non-patent Literature 7), and there is a concern that the agent increases the hotbeds of cancer stem cell formation. Accordingly, in a treatment targeting angiogenesis, therapeutic agents for destroying mature blood vessels are needed in addition to conventional antiangiogenic agents for destroying immature blood vessels.

### CITATION LIST

### Non-patent Literature

NPL 1: Ferrara N, Hillan K J, Novotny W. Bevacizumab (Avastin), a humanized anti-VEGF monoclonal antibody for cancer therapy. Biochem Biophys Res Commun. 2005 Jul 29; 333(2):328-335.

NPT 2: Satoh N, Yamada Y, Kinugasa Y and Takakura N. Angiopoietin-1 alters tumor growth by stabilizing blood vessels or by promoting angiogenesis. Cancer Sci. 99:2373-2379, 2008.

NPL 3: Tozer G M, Kanthou C, Baguley B C. Disrupting tumour blood vessels. Nat. Rev Cancer, 5: 423-435

NPL 4: Paez-Ribes M, Allen E, Hudock J, Takeda T, Okuyama H, Vinals F, Inoue M, Bergers G, Hanahan D, Casanovas O: Antiangiogenic therapy elicits malignant progression of tumors to increased local invasion and distant metastasis. Cancer Cell 15:220-231, 2009.

NPL 5: Ellis L M, Reardon D A: Cancer: The nuances of therapy. Nature 458:290-292, 2009.

NPL 6: Nagahama Y, Ueno M, Miyamoto S, Morii E, Minami T, Mochizuki N, Saya H, Takakura N. 1282796487078_{—}0.Pubmed_RVDocSum&ordinalpos=1 Cancer Res 70, 1215-1224, 2010.

NPL 7: Jain R K: Molecular regulation of vessel maturation. Nat Med 9:685-693, 2003.

### SUMMARY OF INVENTION

### Technical Problem

An object of the present invention is to provide an antiangiogenic agent having a higher treatment effect than those of conventional antiangiogenic agents, and a method for inhibiting angiogenesis using the same. Another object of the present invention is to provide a mature blood vessel-destroying agent in which the agent destroys mature blood vessels, and a method for destroying mature blood vessels using the agent.

### Solution to Problem

To achieve the aforementioned objects, the present inventors focused on the action sites and action mechanisms of an antiangiogenic agent. Specifically, the present inventors assumed that because of a problem in the action site, i.e., a target angiogenesis factor, and in the action mechanism, i.e., inhibiting means, conventional antiangiogenic agents do not attain sufficient treatment effects, and they conducted studies.

With regard to the action site, the following considerations were made. Avastin (registered trademark), for example, targets VEGF; however, when only VEGF is inhibited, since expression of other angiogenesis factors is induced in a compensatory manner, angiogenesis cannot be fully inhibited.

Therefore, the present inventors arrived at the idea of targeting an angiogenesis factor, which cannot be substituted by other angiogenesis factors. In addition, the present inventors focused in particular on an angiogenesis factor involving a lumen formation process. Examples of such angiogenesis factors include VE-cadherin, Claudin-5, and the like.

With regard to the action mechanisms, the following considerations were made. For example, although Avastin (registered trademark) acts by inhibiting the activity of an angiogenesis factor, higher effects could be attained if the target is inhibited in a protein expression stage. Then, the present inventors conceived of using micro RNA (miRNA).

However, there have been few reports on miRNA that inhibits an angiogenesis factor. Moreover, processes involving previously determining a target protein and searching a miRNA having an inhibition effect on the target protein are generally extremely difficult. The present inventors conducted laborious trial and error, and accidentally found that, among miRNAs, miRNA125b (miR125b) inhibits the expression of VE-cadherin to thereby inhibit angiogenesis. Further, the present inventors found that the use of miR125b actually attains an angiogenesis inhibitory effect and a cancer metastasis inhibitory effect. Moreover, the present inventors found that miR125b can destroy mature blood vessels in a focus site in which mature blood vessels of cancer tissues, etc., are formed, to inhibit oxygen delivery to the focus site; and that miR125b is effective for curing the focus site. The present inventors conducted research based on these findings, and accomplished the present invention.

Specifically, the present invention is as follows:

1. An antiangiogenic agent comprising at least one miRNA type selected from the group consisting of miRNAs, pre-miRNAs, and pri-miRNAs, each having a miRNA activity on VE-cadherin; or comprising a recombinant vector including polynucleotide encoding the miRNA type.
2. The antiangiogenic agent according to Item 1, wherein the miRNA type exhibits a miRNA activity by binding to the region of the base sequence represented by SEQ ID No. 1 of mRNA encoding VE-cadherin.
3. The antiangiogenic agent according to Item 2, wherein a portion constituting miRNA in a base sequence of the miRNA type includes a base sequence represented by SEQ ID No. 2.
4. The antiangiogenic agent according to Item 3, wherein a portion constituting miRNA in the base sequence of the miRNA type includes a base sequence in which the following (A) or (B) binds to the 3′ terminal end of a base sequence represented by SEQ ID No. 2:
(A) a base sequence represented by SEQ ID No. 3; or
(B) a base sequence represented by SEQ ID No. 3 in which one or a plurality of nucleotides is deleted, substituted, or added.
5. The antiangiogenic agent according to Item 1, wherein the number of bases of a portion constituting miRNA in the base sequence of the miRNA type is 19 to 25.
6. The antiangiogenic agent according to Item 1, which is used as an agent for treating inflammation diseases.
7. The antiangiogenic agent according to Item 1, which is used as an agent for treating age-related macular degeneration.
8. The antiangiogenic agent according to Item 1, which is used as an agent for inhibiting cancer metastasis or cancer invasion.
9. An agent for destroying a mature blood vessel, comprising at least one miRNA type selected from the group consisting of miRNAs, pre-miRNAs, and pri-miRNAs, each having a miRNA activity on VE-cadherin; or comprising a recombinant vector containing polynucleotide encoding the miRNA type.
10. The agent according to Item 9, which is used as an agent for treating solid cancers or chronic inflammation diseases.
11. A method for inhibiting angiogenesis in a disease that is caused or worsens because of angiogenesis, comprising the step of administering the antiangiogenic agent according to Item 1 in an amount effective for treatment to a patient having the disease.
12. A method for inhibiting cancer metastasis or cancer invasion, comprising the step of administering the antiangiogenic agent according to Item 1 in an amount effective for treatment to a cancer patient in need of inhibition of cancer metastasis or cancer invasion.
13. A method for destroying a mature blood vessel formed in a focus, comprising the step of administering the agent for destroying a mature blood vessel according to Item 9 in an amount effective for treatment to a patient having the focus in which the mature blood vessel is formed.
14. Use of at least one miRNA type selected from the group consisting of miRNAs, pre-miRNAs, and pri-miRNAs, each having a miRNA activity on VE-cadherin, or a recombinant vector comprising polynucleotide encoding the miRNA type for the production of an antiangiogenic agent.
15. Use of at least one miRNA type selected from the group consisting of miRNAs, pre-miRNAs, and pri-miRNAs, each having a miRNA activity on VE-cadherin, or a recombinant vector comprising polynucleotide encoding the miRNA type for the production of an agent for destroying a mature blood vessel.

### Advantageous Effects of Invention

The antiangiogenic agent of the present invention has an angiogenesis inhibitory effect. In particular, the antiangiogenic agent of the present invention has (i) an effect of inhibiting the growth of endothelial cells, (ii) an effect of inhibiting lumen formation by endothelial cells, and (iii) an effect of inhibiting movement of endothelial cells.

The antiangiogenic agent of the present invention has an angiogenesis inhibitory effect, thereby attaining an effect of inhibiting cancer metastasis and invasion. Further, the antiangiogenic agent of the present invention has an effective treatment effect on inflammation diseases and age-related macular degeneration diseases, based on the effect of inhibiting angiogenesis.

The agent for destroying a mature blood vessel of the present invention can destroy a mature blood vessel in a focus site in which a mature blood vessel of a tumor tissue, etc., is formed. Therefore, by inhibiting oxygen delivery to the focus site, the focus can be cured.

### BRIEF DESCRIPTION OF DRAWINGS

FIG. 1 shows the stem-loop sequence (top) and mature sequence (bottom) of miR125b. The underlined part in the mature sequence is a seed sequence of a functional region of the miR125b.

FIG. 2 is a graph showing the results of the expression amount of miR125b in endothelial cells in which angiogenesis occurs, the expression amount being examined using real-time quantitative PCR.

FIG. 3 is a graph showing the results of the expression amount of miR125b in VEGF-stimulated endothelial cells, the expression amount being examined using real-time quantitative PCR.

FIG. 4 is a graph showing the results of the expression amount of miR125b in demethylated endothelial cells, the expression amount being examined using real-time quantitative PCR.

FIG. 5 is a graph showing the examined results of the cell-growth speed of miR125b-transfected vascular endothelial cells.

FIG. 6 shows photographs used in place of drawings, in which the lumen formation of control cells (non-transfected vascular endothelial cells) and miR125b-transfected vascular endothelial cells is photographed.

FIG. 7 shows photographs used in place of drawings, showing cell movement ability of control cells (non-transfected vascular endothelial cells) and miR125b-transfected vascular endothelial cells.

FIG. 8 is a graph showing the results in which change in the expression amount of VE-cadherin and claudin-5 by miR125b transfection is measured using a comparative threshold cycle method.

FIG. 9 shows photographs used in place of drawings, showing the results in which change in the expression amount of VE-cadherin and claudin-5 by miR125b transfection is evaluated using antibody staining.

FIG. 10 shows a photograph of mice in which the function of miR125b in cancer mice is analyzed. The left shows a tumor image of a control in which only a transfection reagent is injected, the middle shows a tumor image of a mouse in which miR125b is transfected, and the right shows a tumor image of a mouse in which miR125 and anti-miR, which is a complementary strand of miR125b, are transfected.

FIG. 11 is a graph showing a tumor growth-inhibiting effect in a control in which only a transfection reagent is injected, a mouse in which miR125b is transfected, and a mouse in which miR125b and anti-miR, which is a complementary strand of miR125b, are transfected.

FIG. 12 shows photographs used in place of drawings, showing the results of the expression of VE-cadherin in vascular endothelial cells in the control in which only a transfection reagent is injected, and in the mouse in which miR125b is transfected, the expression being evaluated using antibody staining.

FIG. 13 shows photographs used in place of drawings, showing the evaluation results of the presence and hypoxic conditions of CD31 positive vascular endothelial cells in the control in which only a transfection reagent is injected, and of the mouse in which miR125b is transfected.

FIG. 14(A) shows blood vessels formed in Matrigel, which are stained with a CD31 antibody. The blood vessels are in green.

FIG. 14(B) shows data in which the number of blood vessels formed in the Matrigel is calculated. The numerical data (Y axis) is a relative value obtained by calculating the number of blood vessels induced by miR125b, considering that the number of blood vessels induced by VEGF, bFGF, or vehicle is 1.

FIG. 15(A) shows the results of choroid observation in which premicroRNA125b or control premicroRNA is administered to an age-related macular degeneration model. The angiogenesis region newly formed in the retina is enclosed by the dashed line. Blood vessels are stained with dextran.

FIG. 15(B) shows the results in which the blood vessel region (CNV area) formed in the retina is measured.

FIG. 16 is a drawing showing the effect of miR125b. The blood vessel has a cord structure in which endothelial cells are gathered, and the endothelial cells are adhered to each other using VE-cadherin to form a lumen (tube). miR125b inhibits protein translation of VE-cadherin, thereby inhibiting lumen formation.

### DESCRIPTION OF EMBODIMENTS

### A. Antiangiogenic Agent

The antiangiogenic agent of the present invention comprises at least one miRNA type selected from the group consisting of miRNAs, pre-miRNAs, and pri-miRNAs, each having a miRNA activity on VE-cadherin; or comprises a recombinant vector containing a gene encoding the miRNA type.

1. miRNA

A miRNA is a short, endogenous, single-stranded RNA that is not translated into a protein. A miRNA targets a specific mRNA, inhibits protein translation of the target mRNA, and/or destabilizes the target mRNA. A miRNA acts when binding to its complementary base sequence in the target mRNA.

The most well-known miRNA is a miRNA that is 22 nucleotides in length. In the present invention, miRNAs that are 19 to 25 nucleotides in length can be used. In particular, miRNAs that are 20 to 24 nucleotides in length are preferably used, and miRNAs that are 21 to 23 nucleotides in length are more preferably used.

A pre-miRNA is an abbreviation of precursor miRNA, and is a precursor of miRNA. A pre-miRNA is a double-stranded RNA having a stem-loop structure including a miRNA sequence in a stem portion. The miRNA sequence is cleaved from the pre-miRNA by an enzyme called Dicer.

The most well-known pre-miRNA is a pre-miRNA that is about 70 nucleotides in length. In the present invention, miRNAs that are 60 to 800 nucleotides in length can be used. In particular, miRNAs that are 70 to 200 nucleotides in length are preferably used, and miRNAs that are 80 to 100 nucleotides in length are more preferably used.

A pri-miRNA is an abbreviation of primary miRNA, and is a primary transcript transcribed from a genome. A pri-miRNA is a double-stranded RNA having a cap structure, a poly(A)tail, and a stem-loop structure including a miRNA sequence in the stem portion. A pre-miRNA is formed by cleaving part of pri-miRNA with an enzyme called Drosha. Pri-miRNAs that are hundreds to thousands of nucleotides in length are known.

As a miRNA type, miRNAs, pre-miRNAs, and pri-miRNAs can be used singly, or in a combination of two or more.

2. miRNA Activity on VE-cadherin

miRNA activity is an activity in which a miRNA type inhibits protein translation of the target mRNA. Specifically, when miRNA is used as a miRNA type, miRNA activity indicates an activity in which miRNA acts on the target mRNA to inhibit protein translation of the target mRNA. When pre-miRNA or pri-miRNA is used as a miRNA type, miRNA activity indicates an activity in which miRNA resulting from pre-miRNA or pri-miRNA acts on the target mRNA to inhibit protein translation of the target mRNA.

Whether a certain type of miRNA has a miRNA activity on VE-cadherin can be determined by checking whether the expression of VE-cadherin in human umbilical vein endothelial cells (HUVECs) in which the miRNA is introduced is inhibited. When the expression is inhibited, the miRNA is considered to have a miRNA activity; and when the expression is not inhibited, the miRNA is not considered to have a miRNA activity. The miRNA type is introduced in accordance with the description of Test Example 2, under the conditions such that a sufficient amount of the miRNA type is introduced.

3. miRNA Type Having a miRNA Activity on VE-cadherin

Examples of the miRNA type having a miRNA activity on VE-cadherin include the following. For example, miRNAs that exhibit a miRNA activity by binding to a base sequence present in the 3′ untranslated region of mRNA encoding VE-cadherin can be used. Specifically, miRNAs that exhibit a miRNA activity by binding to the region of the base sequence represented by SEQ ID No. 1, which is a partial sequence of mRNA encoding VE-cadherin, can be used. Further, pre-miRNAs and pri-miRNAs, each producing the miRNA, can also be used.

The base sequence represented by SEQ ID No. 1 is a base sequence that is present in the 3′ untranslated region of mRNA encoding VE-cadherin.

Examples of the miRNA type that exhibits a miRNA activity by binding to the base sequence represented by SEQ ID No. 1 include miRNAs having the base sequence represented by SEQ ID No. 2; and pre-miRNAs or pri-miRNAs, each producing the miRNA. The pre-miRNAs or pri-miRNAs, each producing the miRNA, are those in which a portion constituting a stem structure in the base sequence of the pre-miRNAs or pri-miRNAs, i.e., a portion constituting the miRNA, includes the base sequence represented by SEQ ID No. 2.

The base sequence represented by SEQ ID No. 2 is a base sequence complementary to the base sequence represented by SEQ ID No. 1.

The miRNA type that exhibits a miRNA activity by binding to the base sequence represented by SEQ ID No. 1 is preferably a miRNA having the base sequence represented by SEQ ID No. 2 at the 5′ terminal side; or a pre-miRNA or pri-miRNA, each producing the miRNA.

The miRNA type that exhibits a miRNA activity by binding to the base sequence represented by SEQ ID No. 1 is more preferably a miRNA having a base sequence in which the base sequence represented by SEQ ID No. 3 is linked to the 3′ terminal side of the base sequence represented by SEQ ID No. 2, or a pre-miRNA or pri-miRNA, each producing the miRNA. A preferable example of the miRNA having a base sequence in which the base sequence represented by SEQ ID No. 3 binds to the 3′ terminal side of the base sequence represented by SEQ ID No. 2 is miRNA125b (miR125b), which is a miRNA having a base sequence in which the base sequence represented by SEQ ID No. 3 is linked to the 3′ terminal side of the base sequence represented by SEQ ID No. 2.

There has been no report indicating that angiogenesis is inhibited by miR125b.

Preferable examples of the miRNA type that exhibits a miRNA activity by binding to the base sequence represented by SEQ ID No. 1 include those having a base sequence obtained by linking a base sequence in which one or a plurality of nucleotides is deleted, substituted, or added in the base sequence represented by SEQ ID No. 3 to the 3′ terminal side of the base sequence represented by SEQ ID No. 2, and having a miRNA activity; and pre-miRNAs or pri-miRNAs, each producing RNA comprising the aforementioned base sequence.

As the “base sequence in which one or a plurality of nucleotides is deleted, substituted, or added,” a base sequence in which 1 to 9 nucleotide(s) is deleted is preferable, a base sequence in which 1 to 5 nucleotide(s) is deleted is more preferable, and a base sequence in which 1 to 4 nucleotide(s) is deleted is particularly preferable. Further, base sequences in which 1 to 4, particularly 1 to 3, and more particularly 1 or 2 nucleotide(s) is deleted are preferable.

4. Recombinant Vector Containing a Gene Encoding a miRNA Type having a miRNA Activity on VE-cadherin

A recombinant vector containing a gene encoding a miRNA type having a miRNA activity on VE-cadherin (hereinafter sometimes referred to as “miRNA type recombinant vector”) is a recombinant vector that exhibits the same miRNA activity as that explained in the miRNA type section above, by the expression of the miRNA in cells.

The recombinant vector can be produced by introducing DNA encoding the miRNA type into an appropriate expression vector.

The recombinant vector includes, in addition to a DNA encoding the aforementioned miRNA type, a base sequence that controls the expression. Examples of the base sequence include a promoter sequence located on the upstream side of the DNA encoding the miRNA type.

Examples of the promoter sequence include CMV, EF1, etc.

In addition to the above, it is possible to provide flanking regions in the 5′ and 3′ sides of the target miRNA across the miRNA.

Usable examples of the expression vector include lentiviral vectors, adenovirus vectors, non-wrench virus vectors, non-adenovirus vectors , etc.

The DNA can be introduced by a known method. For example, after an expression vector is cleaved with a single or a plurality of restriction enzyme(s), the DNA can be introduced into the cleaved portion. If necessary, the DNA can be introduced after being cleaved with the same or different restriction enzyme(s). It is also possible to introduce the DNA through a linker. If necessary, the DNA may be introduced after a protruding end portion generated by a restriction enzyme treatment is blunt ended.

### 5. Method for Administering an Antiangiogenic Agent

The antiangiogenic agent of the present invention can be administered according to a method that is generally used as a method for introducing a gene or oligonucleotide (transfection). The administration form of the antiangiogenic agent is suitably determined depending on a target area, etc. The agent may be systemically or locally administered. Examples of the systemic administration include oral administration and parenteral administration. Examples of the parenteral administration include intravenous injection, subcutaneous injection, intramuscular injection, etc. The antiangiogenic agent of the present invention can be locally administered to a skin, mucosa, respiratory tract, abdominal cavity, nose, eyes, brain, or the like.

The form of the antiangiogenic agent of the present invention is suitably determined according to the administration form. Examples thereof include tablets, granules, powders, suppositories, capsules or like solid forms; creams, gels, ointments or like semi-solid forms; liquids, lotions or like liquid forms, etc.

The dose and frequency of the administration of the antiangiogenic agent of the present invention are suitably determined according to the administration form, form of the agent, conditions of a person to be administered, degree of miRNA activity of a miRNA type, expression efficiency of a recombinant vector, etc. The dose of the administration is not limited, as long as it is effective in a treatment. For example, the average dose is preferably 0.00001 to 200 mg/kg, more preferably 0.2 to 15 mg/kg, and even more preferably 1 to 2.5 mg/kg, per day, per kg of the body weight, on a miRNA basis.

In the case where the antiangiogenic agent is a miRNA type as an active component, “on a miRNA basis” means an antiangiogenic agent including the predetermined amount of miRNA when the miRNA type is miRNA, and means an antiangiogenic agent including pre-miRNA or pri-miRNA, each being capable of producing the predetermined amount of miRNA, when the miRNA type is pre-miRNA or pri-miRNA. In the case where the antiangiogenic agent includes a miRNA type recombinant vector as an active component, “on a miRNA basis” means an antiangiogenic agent including a recombinant vector expressing the predetermined amount of miRNA when the miRNA type is miRNA, and means an antiangiogenic agent including a recombinant vector expressing pre-miRNA or pri-miRNA, each being capable of producing the predetermined amount of miRNA, when the miRNA type is pre-miRNA or pri-miRNA. The antiangiogenic agent of the present invention may be administered in 1 dose per day, or about 2 to 3 doses per day. It is also possible to administer a 2- to 7-day-agent dosage amount at one time.

The proportion of a miRNA type recombinant vector or a miRNA type in the miRNA type-containing antiangiogenic agent of the present invention is determined according to the administration form, form of the agent, dose, dose frequency, etc.

The antiangiogenic agent of the present invention may optionally contain, in addition to the active component (miRNA type or a miRNA type recombinant vector), a pharmacologically acceptable carrier and/or other medicinal components. Examples of the pharmacologically acceptable carrier include transfection reagents, preparation components necessary for forming a preparation according to the form of the agent, storage-stable components necessary for stable storage, and the like. Of these carriers, transfection reagents are preferably used for efficiently performing the intracellular transfer of the active component (miRNA type or a miRNA recombinant vector) in a target administration portion. As a transfection reagent, those containing a cationic water-soluble polymer or a cationic lipid as a main active component can be used. As a transfection reagent, those containing a virus particle can also be used. Examples of other medicinal components include anti-inflammatory agents, antimicrobial agents, and the like.

### 6. Antiangiogenic Agent Administration Subjects

By administering the antiangiogenic agent of the present invention to a patient having a disease that is caused or worsens because of angiogenesis, the disease can be treated.

Examples of the disease caused by angiogenesis include diabetic retinopathy, age-related macular degeneration, retinopathy of prematurity, glaucoma, arteriovenous malformation, hemangioma, and the like (1: Pandya N M, Dhalla N S, Santani D D. Angiogenesis, a new target for future therapy. Vascul Pharmacol. 2006 May; 44(5): 265-74. Epub 2006 Mar 20).

Examples of the disease that worsens because of angiogenesis include solid cancers, infectious diseases, arteriosclerosis, various autoimmune diseases such as rheumatoid arthritis and scleroderma, diabetic retinopathy, age-related macular degeneration, retinopathy of prematurity, glaucoma, arteriovenous malformation, hemangioma, degenerative arthritis, keloid, psoriasis, allergic dermatitis, obesity, pulmonary hypertension, asthma, vesicular emphysema, chronic bronchitis, liver cirrhosis, ascites, and the like (Pandya N M, Dhalla N S, Santani D D. Angiogenesis a new target for future therapy. Vascul Pharmacol. 2006 May; 44(5): 265-74. Epub 2006 Mar 20., Buysschaert I, Schmidt T, Roncal C, Carmeliet P, Lambrechts D. Genetics, and epigenetics and pharmaco-(epi) genomics in angiogenesis. J Cell Mol Med. 2008 Dec; 12(6B): 2533-51, Carmeliet P. Angiogenesis in life, and disease and medicine. Nature. 2005 Dec 15; 438(7070): 932-6).

The administration of the antiangiogenic agent is considered effective for these diseases, and the effectiveness of the administration of the antiangiogenic agent is actually reported. (Regarding cancer, see Ferrara N, Hillan K J, Novotny W. Bevacizumab (Avastin), a humanized anti-VEGF monoclonal antibody for cancer therapy, Biochem Biophys Res Commun. 2005, Jul 29; 333 (2): 289-91.; and regarding retinopathy, see Jardeleza M S, Miller J W. Review of anti-VEGF-therapy-in-proliferative-diabetic retinopathy. Semin Ophthalmol. 2009 Mar-Apr; 24 (2):87-92. Of these diseases, the antiangiogenic agent of the present invention has an excellent treatment effect on age-related macular degeneration. Therefore, age-related macular degeneration is a preferable example. When the antiangiogenic agent of the present invention is used for treating a cancer, lung cancer is a suitable example.

Further, since the antiangiogenic agent of the present invention can effectively inhibit angiogenesis induced by inflammation, the antiangiogenic agent of the present invention can be suitably used for the treatment of inflammatory diseases, such as allergic dermatitis and chronic bronchitis.

### 7. Cancer Metastasis- or Cancer Invasion-inhibiting Agent

The action mechanism of the antiangiogenic agent of the present invention is that angiogenesis is inhibited by negatively controlling the expression of VE-cadherin, which plays an essential part in the lumen formation of vascular endothelial cells.

In cancer treatment, metastasis and invasion of a cancer cell often become a problem. It is said that metastasis of a cancer cell requires angiogenesis. By administering the antiangiogenic agent of the present invention to a site where metastasis or invasion of a cancer cell is concered, angiogenesis at that site can be inhibited. As a result, metastasis or invasion of cancer cells can also be inhibited. Therefore, the antiangiogenic agent of the present invention can be used as a cancer metastasis- or cancer invasion-inhibiting agent.

When the antiangiogenic agent of the present is used as a cancer metastasis- or cancer invasion-inhibiting agent, the administration form, form of the agent, dose, and dose frequency are the same as those of the antiangiogenic agent.

### B. Agent for Destroying a Mature Blood Vessel

At least one miRNA type selected from the group consisting of miRNAs, pre-miRNAs, and pri-miRNAs, each having a miRNA activity on VE-cadherin, can destroy mature blood vessels in a focal site to inhibit oxygen delivery to the focal site, thereby curing the focal site. Accordingly, the present invention also provides an agent for destroying a mature blood vessel containing at least one miRNA type selected from the group consisting of miRNAs, pre-miRNAs, and pri-miRNAs, each having a miRNA activity on VE-cadherin, or a recombinant vector containing a gene encoding the miRNA type.

The agent for destroying a mature blood vessel of the present invention is effective for destroying the mature blood vessel formed in the focal site of chronic inflammation diseases such as solid cancers and rheumatoid arthritis, and can be used for curing the focal site.

The administration form, form of the agent, dose, dose frequency, etc., of the agent for destroying a mature blood vessel are the same as those of the antiangiogenic agent.

### EXAMPLES

Hereinafter, the present invention is specifically explained based on the Test Examples and Examples. However, the present invention is not limited to these examples.

### Test Example 1

### Search for miRNA that Increases Expression During Angiogenesis

The present inventors hypothesized that a vascular endothelial cell includes a miRNA that plays a role in inhibiting angiogenesis. Specifically, they assumed that the expression of the miRNA is increased in a cell in which angiogenesis is activated. This hypothesis was verified as follows.

First, endothelial cells were collected from a tumor tissue in which angiogenesis actively occurred. By comparing the collected vascular endothelial cells with vascular endothelial cells in a normal tissue, miRNAs whose expression was increased in the former cells were profiled. 18 types of miRNAs in total were obtained.

Screening results indicated, as shown in Test Examples 2 to 6, that miR125b had (i) an effect of inhibiting the growth of endothelial cells, (ii) an effect of inhibiting lumen formation by endothelial cells, and (iii) an effect of inhibiting movement of endothelial cells, each of them being an effect necessary for inhibiting angiogenesis. Other miRNAs obtained in the screening were not examined hereinbelow because of unstable expression.

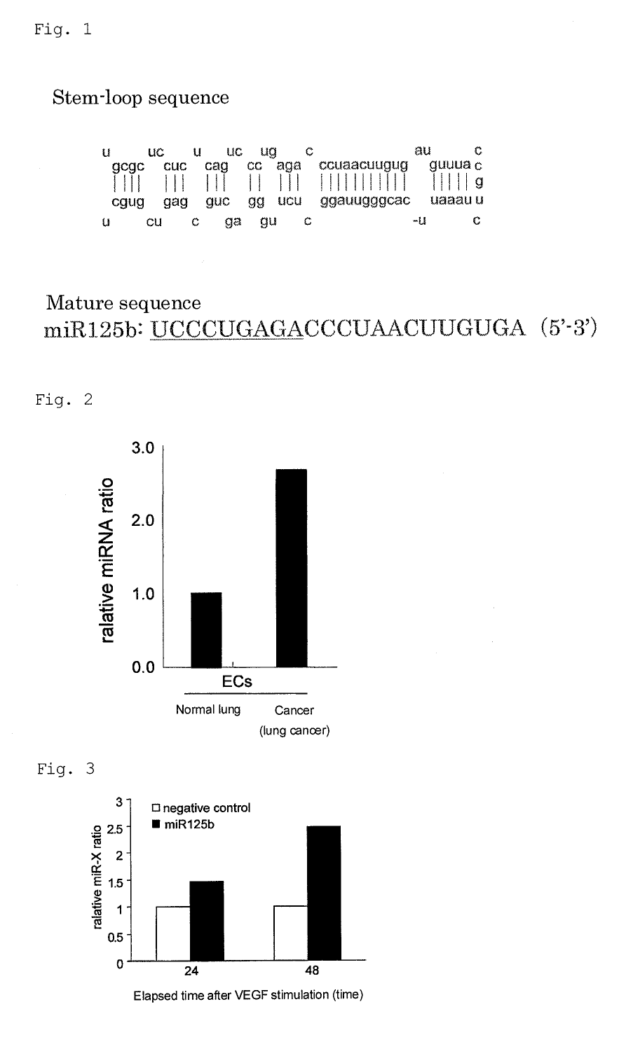

It was found that, in vascular endothelial cells undergoing angiogenesis in a tumor, an about threefold miR125b expression increase was shown compared to endothelial cells in a normal subcutaneous tissue. FIG. 2 shows the results, and the experiment was performed as follows.

### Experimental Method

One of the angiogenesis models is a model that inserts into a tumor formed by implanting a tumor cell under the skin of a mouse, to form a blood vessel. Vascular endothelial cells which are negative to CD45 (blood marker) and positive to CD31 (vascular endothelial cell marker), were collected from a tumor formed by implanting a Lewis lung carcinoma cell into a mouse, and the expression of miR125b was analyzed. After washing the collected endothelial cells with PBS, miRNAs were collected according to the instructions of PureLink miRNA Isolation Kit (Invitrogen, K1570-01). The collected miRNAs were stored at −80° C., and then the reverse transcription of miRNAs was carried out according to the instructions of NCode miRNA First-Strand cDNA Synthesis and qRT-PCR Kits (Invitrogen, MIRC-50). The real-time quantitative PCR was performed using SYBR Green ER qPCR SuperMix Universal (Invitrogen, 11762-100). The primer sequence for miR125b was 5′-CCC TGA GAC CCT AAC TTG TGA-3′, and the primer sequence for RNU6 was 5′-CGC TTC GGC AGC ACA TAT AC-3′, 5′-AAA ATA TGG AAC GCT TCA CGA-3′. The amount of miRNA was analyzed by standardizing the amount of each miRNA using RNU6 miRNA as an endogenous control, and using a comparative threshold cycle method.

In the endothelial cells, it was confirmed that the expression of miR125b was induced by VEGF stimulation. FIG. 3 shows the results, and the experiment was performed as follows.

### Experimental Method

Human umbilical vein endothelial cells (HUVECs) (2×10⁵) were seeded on a 6-well plate. 20 ng/ml VEGF-A165 (PeproTech, Co., Ltd.) was added to HuMedia-EG2, and cultured in a CO₂ incubator at 37° C. 24 hours and 48 hours later, the HUVECs were washed with PBS, and miRNAs were collected according to the instructions of PureLink miRNA Isolation Kit (Invitrogen). As in FIG. 2, analysis was performed using real-time quantitative PCR.

It was further confirmed that the expression of miR125b was induced by demethylation in the endothelial cells. FIG. 4 shows the results, and the experiment was performed as follows.

### Experimental Method

5-Aza-2′-deoxycytidine (SIGMA) was solubilized with PBS to prepare a 10 mM 5-Aza-dC solution. 2×10⁵ HUVECs were seeded on a 6-well plate, and a 10 mM 5-Aza-dC solution was added to HuMedia-EG2 to form a 0.2 μM, 2 μM, 20 μM, or 200 μM solution, followed by culturing in a CO₂ incubator at 37° C. 24 hours later, the HUVECs were washed with PBS, and miRNAs were collected according to the instructions of PureLink miRNA Isolation Kit (Invitrogen). As in FIG. 2, analysis was performed using real-time quantitative PCR.

### Test Example 2

### Effect of miR125b for Inhibiting Endothelial Cell Growth

Regarding the relation between miR125b and angiogenesis, effects on the vascular endothelial cell growth were observed.

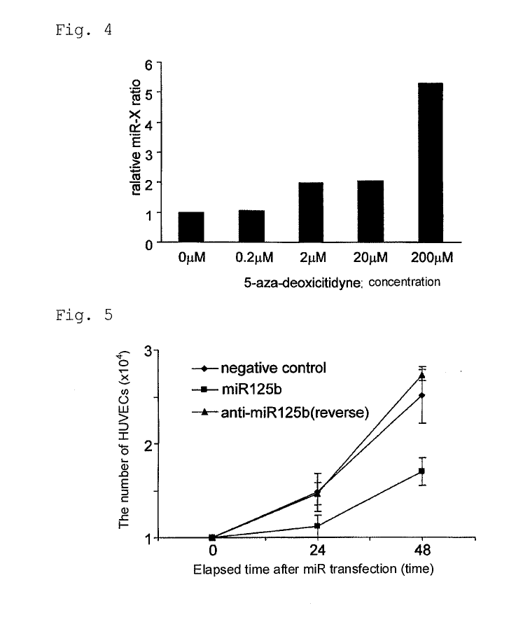

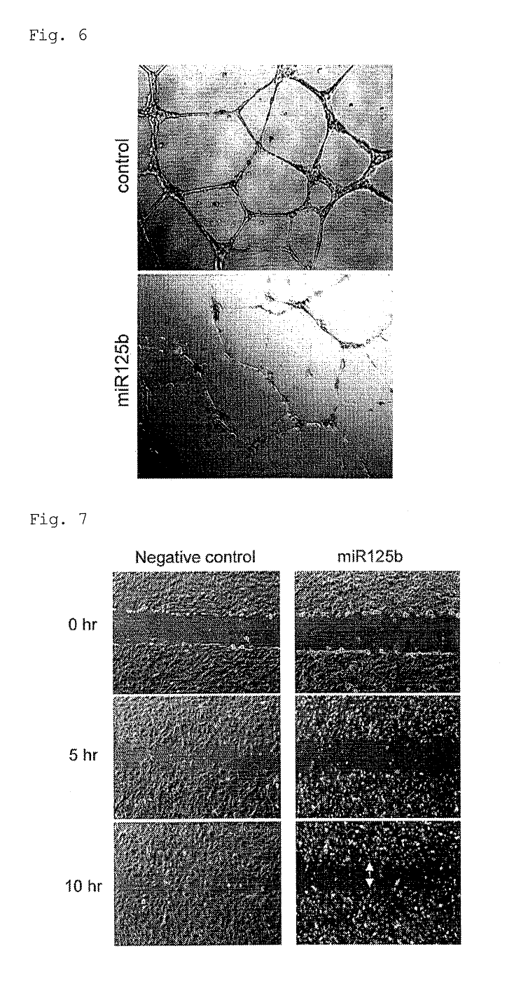

HUVECs were cultured in a complete medium in which a HuMedia-MvG growth additive set was added to a basic culture medium HuMedia-EB2. HUVECs which were subcultured four to seven times were seeded on a plate, and on the next day, 30 to 50% confluent state of HUVECs on the plate was confirmed. The culture medium was changed from the complete medium to an antibiotic-free culture medium (HuMedia-EB2+2%FBS), and culturing was performed at 37° C. in a CO₂ incubator for 1 hour. The miRNA precursor was transfected according to the instructions of Lipofectamine 2000 Reagent (Invitrogen) so that the final concentration became 33 mM. As transfection reagents, Lipofectamine 2000 Reagent (Invitrogen) and Opti-MEMI Reduced Serum Medium (GIBCO) were used. As miRNA precursors, Negative Control #1 (AM17110), Pre-miR Precursor (PM10148), and Anti-miR Inhibitor (AM10148) were purchased from Applied Biosystems. The HUVECs, HuMedia-EB2 (basic culture medium), and a HuMedia-MvG growth additive set were purchased from Kurabo Industries, Ltd. On the next day, HUVECs which was transfected with a miRNA precursor were seeded on a 96-well plate so as to have 1×10⁴ cells/100 24 hours and 48 hours later, cells were collected after trypsin treatment, and the number of cells was counted using a blood cell counting board. The results indicated, as shown in FIG. 5, that miR125b inhibited cell growth of the vascular endothelial cells.

### Test Example 3

### Effect of miR125b for Inhibiting Lumen Formation of Endothelial Cells

150 μl of Growth Factor Reduced BD Matrigel Matrix (BD Bioscience) was placed on a 48-well plate, and the plate was placed in a CO₂ incubator at 37° C. for 30 minutes to solidify the Matrigel. Thereafter, as shown in FIG. 3, HUVECs which was transfected with a miRNA precursor were collected after trypsin treatment. Cells were seeded on the solidified Matrigel so as to have a complete medium, 1×10⁵ cells/150 μl per well, and placed in a CO₂ incubator at 37° C. 5 hours later, the cells were photographed. The results indicated, as shown in FIG. 6, that although the endothelial cells in the control had a lumen-like structure, the lumen formation was inhibited in the endothelial cells into which miR125b was introduced.

### Test Example 4

### Effect of miR125b for Inhibiting the Movement of Endothelial Cells

After confirming that HUVECs, which were transfected with a miRNA precursor, became confluent on a 6-well plate according to the method shown in FIG. 3 on the next day, the bottom of the plate was scratched with the top of the tip of a Pipetman. Immediately after, 5 hours after, and 10 hour after the scratch, the scratched cell regions were photographed. The results indicated, as shown in FIG. 7, that endothelial cells into which miR125b was introduced showed a remarkably lowered cell movement ability.

### Test Example 5

### mRNA Expression Control of VE-cadherin and claudin-5 by miR125b

The aforementioned results suggested that miR125b may affect cell adhesion. To form a lumen using vascular endothelial cells, the expression of VE-cadherin or claudin-5, which plays a role in binding endothelial cells together, is important. Therefore, the possibility of affecting the expression of gene and protein thereof was examined. First, the expression of the gene was analyzed using PCR.

As shown in FIG. 4, endothelial cells into which miR125b was introduced and control endothelial cells were washed with PBS, and mRNAs were collected according to the instructions of RNeasy Mini Kit (QIAGEN). The collected mRNAs were stored at −20° C., and then reverse transcription was carried out according to the instructions of PrimeScript RT Reagent Kit (TAKARA). Real-time quantitative PCR was performed using SYBR Green ER qPCR SuperMix Universal (Invitrogen, 11762-100). The primer sequence for human VE-cadherin was 5′-ATC GGT TGT TCA ATG CGT CC-3′ and 5′-CCT TCA GGA TTT GGT ACA TGA CA-3′, the primer sequence for human claudin-5 was 5 ‘-TCG TTG CGC TCT TCG TGAC-3’ and 5′-CAG CCC GCA AAA CAG GTA G-3′, and the primer sequence for human GAPDH was 5′-GAA GGT GAA GGT CGG AGT C-3′ and 5′-GAA GAT GGT GAT GGG ATT TC-3′. The amount of mRNA was analyzed by standardizing the amount of each mRNA using the mRNA of GAPDH, which is a housekeeping gene, as an endogenous control; and using a comparative threshold cycle method.

As shown in FIG. 8, the results indicated that miR125b did not inhibit the expression of mRNAs of VE-cadherin or claudin-5.

### Test Example 6

### Protein Expression Control of VE-cadherin and claudin-5 by miR125b

As shown in FIG. 4, liquid culture media in which miR125b-introduced endothelial cells or control endothelial cells were cultured were discarded, and HUVECs were washed with PBS one time. A lysis buffer (10 mM Tris, 150 mM NaCl, 5 mM EDTA, 1% NP-40, 2 mM PMSF, and 1 mM Na₃VO₄, and 1/100 volume Protease Inhibitor Cocktail (Nacalai Tesque)) was added thereto, and the mixture was solubilized on ice for 10 minutes. The cell lysate was transferred to an Eppendorf tube, and subjected to centrifugation at 4° C. and 12,000 g for 10 minutes, followed by supernatant collection. As primary antibodies, Mouse anti-Claudin-5 (Invitrogen), Purified Rat Anti-Mouse CD144 (BD Bioscience shrine), anti-human VE-cadherin Antibody, Polyclonal (Bender MedSystems), and mouse anti-GAPDH monoclonal antibody (CHEMICON) were used. As secondary antibodies, Polyclonal Goat Anti-Mouse Immunoglobulins/HRP (Dako), Polyclonal Goat Anti-Rabbit Immunoglobulins/HRP (Dako), Goat Anti-Rabbit Ig's HRP

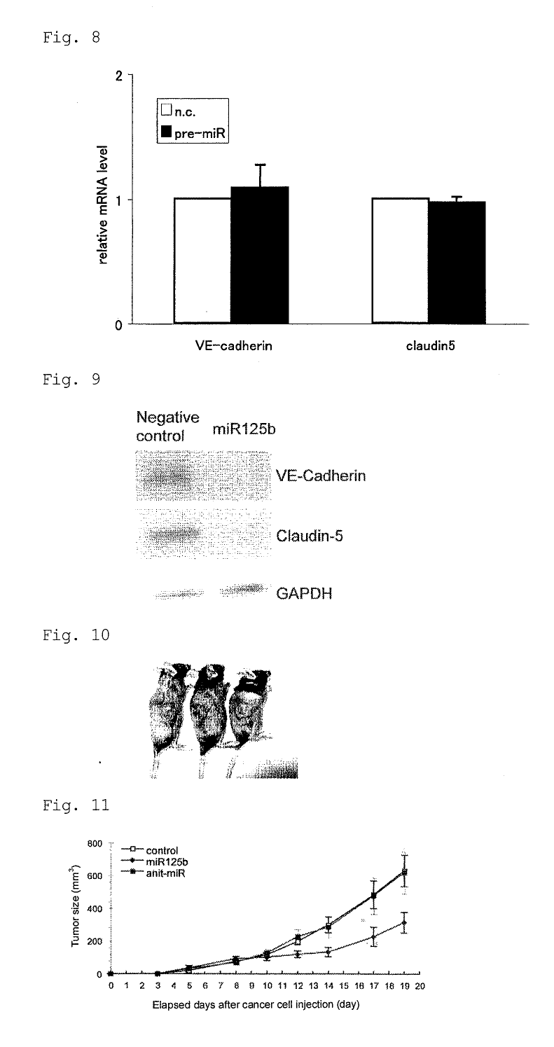

Conjugate (BIOSOURCE), and Goat Anti-Rabbit Ig's HRP (BIOSOURCE) were used. To detect chemiluminescence, LA-3000 mini (FUJIFILM) was used. Although miR125b did not affect the expression of mRNA of VE-cadherin or claudin-5, it inhibited the protein expression of VE-cadherin and claudin-5, as shown in FIG. 9.

### Example 1

### Effect of miR125b for Inhibiting Tumor Growth

The aforementioned results indicate that miR125b may negatively control the expression of junction protein between endothelial cells, which plays an essential part in lumen formation in vascular endothelial cells, thereby inhibiting angiogenesis. Then, whether miR125 can inhibit tumor angiogenesis to thereby inhibit tumor growth was examined.

Lewis lung cancer cells (LLC cells) were collected after trypsin treatment, washed with PBS two times, and subcutaneously injected into 6-week-old C57BL/6 Cr Slc mice (SLC) in such a manner that each had 3×10⁶ cells/200 μl PBS. The size of the tumor was measured every two to three days. 10, 12, 14, and 17 days later, pre-miRNA of miR125b was injected into a tumor using a 26 G syringe in order to perform transfection. As a transfection reagent, in vivo-jetPEI (Polyplus Transfection) was used. 400 pmol of a miRNA precursor and 0.35 μl of in vivo-jet PEI were used per mouse per time to prepare a transfection reagent according to the instructions of Polyplus Transfection. 19 days after the tumor injection, the tumor was collected from each mouse. Part of the tumor tissue was finely cut, and miRNA was collected using miRNA PureLink miRNA Isolation Kit (Invitrogen, K1570-01). The remaining tumor tissue was divided into two, shaken using 4% PFA/PBS at 4° C. for 3 hours, and fixed. After the completion of the fixation, the tumor tissue was shaken using PBS at 4° C. overnight. Thereafter, the tumor tissue was shaken at 4° C. in 15% sucrose/PBS for 3 hours, and shaken at 4° C. in 30% sucrose/PBS for 3 hours. The tumor tissue was then embedded with an O.C.T. compound (Tissue-Tek), placed at −20° C. for 3 hours, and frozen at −80° C. for at least 3 days.

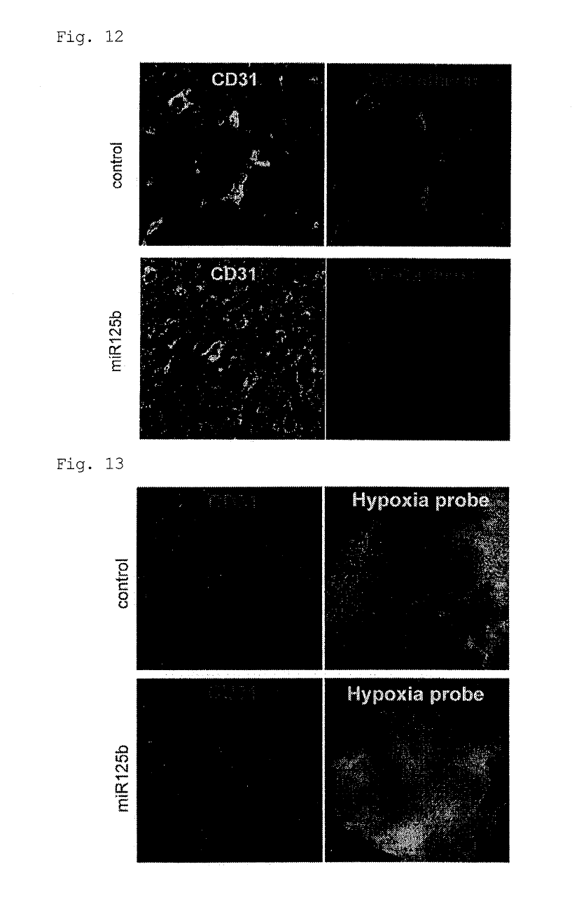

As a result, as shown in FIGS. 10 and 11, significant tumor growth inhibition was observed in the mice to which miR125b was administered, compared to a control into which only a transfection reagent was injected and mice into which anti-miR, which is a complementary strand of miR125b, was injected.

### Example 2

### Effect of miR125b for Inhibiting Expression of VE-cadherin in Endothelial Cells During Angiogenesis

LLC tumor tissues, which were embedded with an O.C.T. compound, were sliced into a 20 μm-segment using a cryostat (LEICA), and the segment was placed on a slide and air-dried for 1 hour using a dryer. The slide was then set in a wash bottle and subjected to one-minute washing 3 times at room temperature using PBS. The O.C.T. compound was dissolved therein. Antigen activation was then performed for 5 minutes using PBST. A liquid blocker was put around the segment, a blocking solution (5% normal goat serum/1%BSA/2% skim milk/PBS) was added dropwise to the segment, and blocking was performed for 30 minutes at room temperature. As a primary antibody, Purified Rat Anti-Mouse CD144 (BD Bioscience 550548) was reacted at 4° C. overnight. After performing a 10 minute-washing 3 times using PBST, secondary antibody Alexa Fluor 647 goat anti-mouse IgG (Molecular Probes) and Alexa Fluor 546 goat anti-rat IgG (Molecular Probes) were reacted at room temperature in the shade for 1 hour. All of the subsequent procedures were performed in the shade at room temperature. Thereafter, 10 minute-washing was performed 3 times using PBST, and a primary antibody Biotin anti-rat CD31 (BD Pharmingen) was reacted for 1 hour. 10 minute-washing was again performed using PBST 3 times, and secondary antibody Streptavidin Alexa Fluor 488 conjugate (Molecular Probes) was reacted for 1 hour. 10 minute-washing was performed 3 times using PBST, several drops of Vectashield (Vector Laboratories, Inc.) were added, and a cover glass was placed thereon. Observation and photographing using a confocal laser microscope were performed.

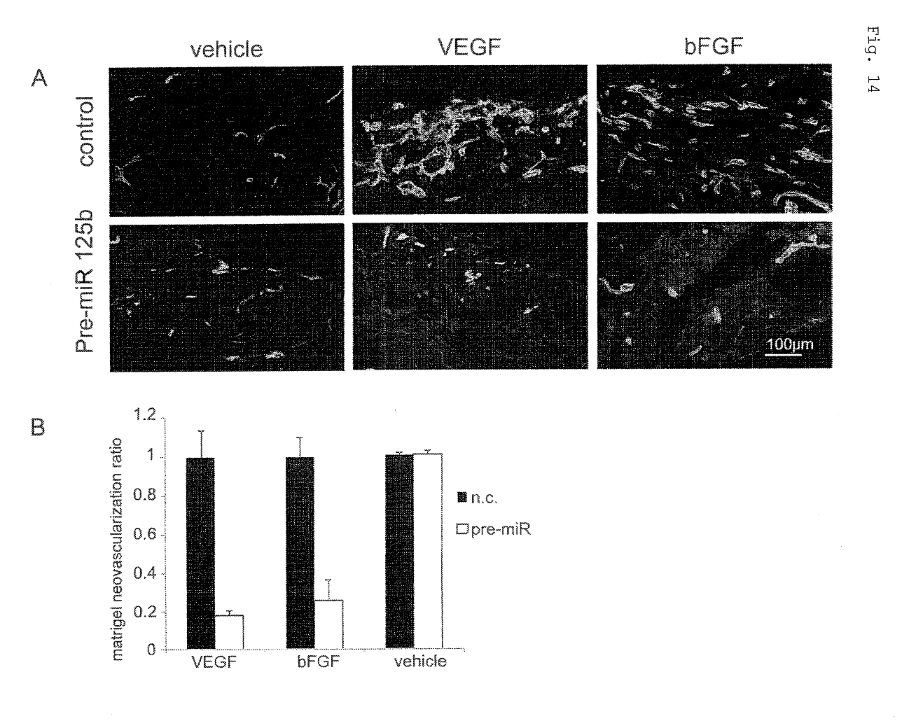

As shown in FIG. 12, the results indicated that although the expression of VE-cadherin was observed in most of the blood vessels of the control tumor, the expression of VE-cadherin was inhibited in vascular endothelial cells of the tumor to which miR125b was administered.

### Example 3

### Induction of Nonfunctional Blood Vessel Formation by miR125b

To observe the blood flow of the blood vessel in the tumor affected by miR125b, analysis was carried out using the degree of tumor hypoxia as an index. Using the tumor segment of FIG. 10, Purified Rat Anti-Mouse-CD31 (BD Pharmingen) and Hypoxyprobe-1 Mabl FITC conjugate (primary antibodies) were reacted overnight in the shade at 4° C. All of the subsequent procedures were performed in the shade at room temperature. 10 minute-washing was performed 3 times using PBST, and a secondary antibody Alexa Fluor 546 goat anti-rat IgG (Molecular Probes) was reacted in the shade at room temperature for 1 hour. Thereafter, 10 minute-washing was again performed 3 times using PBST. Several drops of Vectashield (VECTOR Laboratories Inc.) were added, and a cover glass was placed thereon. Observation and photographing using a confocal laser microscope were then performed.

As shown in FIG. 13, the results indicated that although CD31-positive vascular endothelial cells were present in the tumor to which miR125b was administered, they were hypoxic. The results further indicated that miR125b induces nonfunctional blood vessels that has no blood flow and is in a state where mature blood vessels are destroyed and incapable of delivering oxygen.

### Example 4

### Angiogenesis Inhibition at Inflamed Sites by miR125b

bFGF (basic fibroblast growth factor) and VEGF increase their expression at inflamed sites, thereby inducing angiogenesis. It is well known that inflammation worsens due to angiogenesis. When an extracellular matrix component called Matrigel, including bFGF or VEGF is subcutaneously implanted into a mouse, angiogenesis, which is likely to be observed in inflammation, is induced.

500 μl of each of the following samples was individually administered to the right axilla of female 6-week-old C57BL/6 mice.

A sample in which heparin was added to a BD Matrigel to a final concentration of 30 Unit/ml, and bFGF (R&D systems) was added thereto to a final concentration of 500 ng/ml.
A sample in which heparin was added to a BD Matrigel to a final concentration became 30 Unit/ml, and VEGF (PeproTech) was added thereto to a final concentration of 150 ng/ml.
A sample in which heparin was added to a BD Matrigel to a final concentration of 30 Unit/ml to form a vehicle (PBS alone).
2 Days later and 5 days later, a 3.3 pmol miR125b precursor (pre-miR 125b) was injected into each axilla using in vivo-jetPEI. On day 7, the Matrigel was collected from each mouse. The collected Matrigel was fixed at room temperature in 4 wt % paraformaldehyde (4 wt % paraformaldehyde and 96 wt % PBS) overnight, and embedded with a frozen-tissue embedding agent (OCT compound: Tissue-Tek). Thereafter, a section was formed and a blood vessel in the Matrigel was subjected to immunostaining using a CD31 antibody

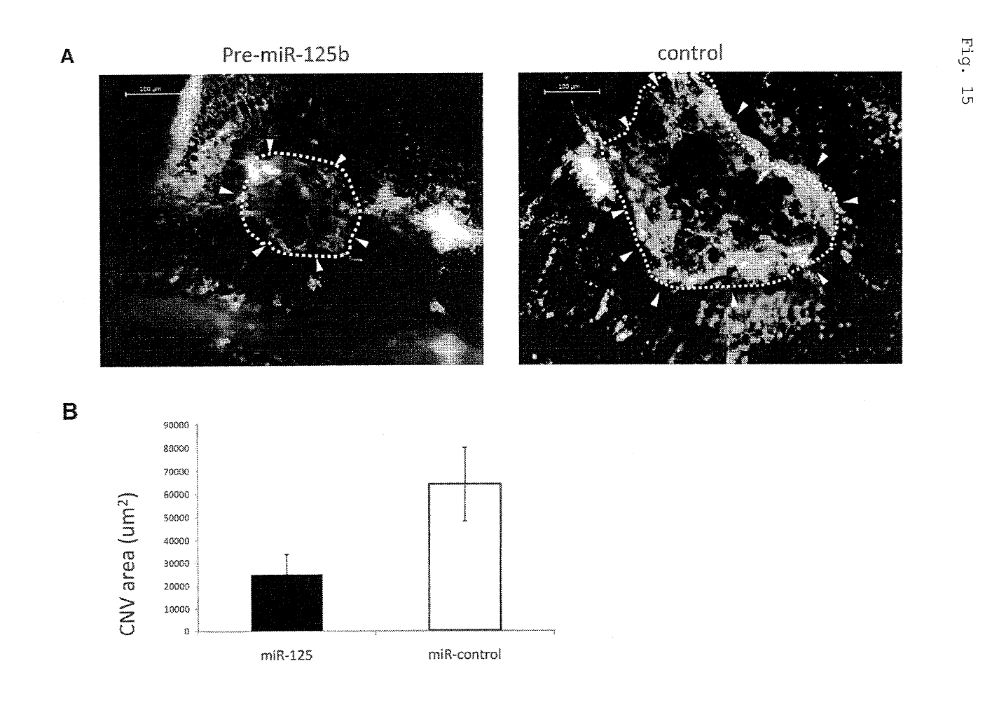

(Pharmingen) and Alexa Fluor 488 Goat Anti-rat IgG (molecular probes) as a secondary antibody. The results indicated that when a miR125b precursor (pre-miR 125b) was not injected, angiogenesis was induced in the Matrigel by VEGF and bFGF (see FIG. 14(A)); however, the angiogenesis induction was inhibited by miR125b (see

FIG. 14 (A) and (B)). As described above, it was revealed that miR125b can inhibit angiogenesis induced by inflammation.

### Example 5

### Angiogenesis Inhibition in Age-related Macular Degeneration Models by miR125b

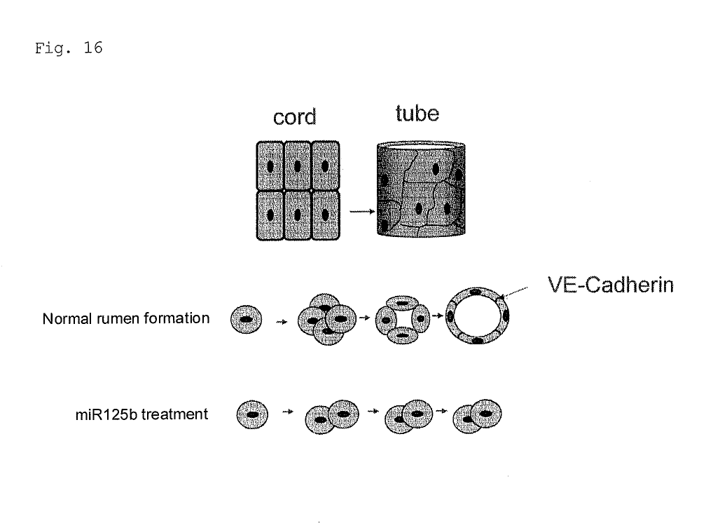

By irradiating laser to the retina of an adult mouse, hyperplasia of abnormal blood vessels is induced in the choroid. This phenomenon occurs with aging, and is used as a model of angiogenesis in age-related macular degeneration, which is considered the main cause of the phenomenon. 30.0 mg/kg pentobarbital anesthesia was intraperitoneally administered to a female 8-week-old wild-type C57BL/6 mouse, and mydriasis was performed by placing a 1% tropicamide drop into each eye. Using an argon green laser, laser-irradiating was performed at four parts around the optic disk of the mouse retina under the conditions of 50 μm and 100 mW for 0.05 seconds so that Bruch membranes were ruptured. Immediately after the laser irradiation, 5 μmol/l of premicroRNA-125 was injected into the vitreous body of the eyeball of the mouse, and a control premicro-RNA having the same concentration was injected into the other eye. The same injection was given 7 days after the laser irradiation. 14 days after the laser irradiation, fluorescein-labeled dextran (molecular probes) having a molecular weight of 2×10⁶ was intravenously injected into the mouse to dye blood vessels beforehand. The eyeballs were extracted, and the whole-mount tissue sections of the choroid were formed. The whole-mount tissue sections were observed and photographed using a fluorescence microscope, and the choroidal neovascular area was measured using blinded subjects. FIG. 15 shows the results. It was revealed that although frequent angiogenesis was induced in a control group, angiogenesis was inhibited by miR125b.

In view of the above, it was revealed that miR125b can inhibit retinal angiogenesis resulting from age-related macular degeneration, etc.

### SEQUENCE LISTING

PCT_antiangiogenic agent and blood_{—}20100826_{—}134003_{—}0.app “0016]

1-15. (canceled)

16. A method for treating a disease that is caused or worsens because of angiogenesis, comprising the step of administering to a patient having the disease an effective amount of an atiangiogeneic agent,
wherein the antiangiogenic agent comprises at least one miRNA type selected from the group consisting of miRNAs, pre-miRNAs, and pri-miRNAs, each having a miRNA activity on VE-cadherin; or comprises a recombinant vector including polynucleotide encoding the miRNA type.

17. A method for inhibiting cancer metastasis or cancer invasion, comprising the step of administering to a patient in need thereof an effective amount of an atiangiogeneic agent, wherein the antiangiogenic agent comprises at least one miRNA type selected from the group consisting of miRNAs, pre-miRNAs, and pri-miRNAs, each having a miRNA activity on VE-cadherin; or comprises a recombinant vector including polynucleotide encoding the miRNA type.

18. A method for destroying a mature blood vessel formed in a focus, comprising the step of administering to a patient in need thereof an effective amount of an atiangiogeneic agent, wherein the antiangiogenic agent comprises at least one miRNA type selected from the group consisting of miRNAs, pre-miRNAs, and pri-miRNAs, each having a miRNA activity on VE-cadherin; or comprises a recombinant vector including polynucleotide encoding the miRNA type.

19. The method according to claim 16, wherein the disease is inflammation.

20. The method according to claim 16, wherein the disease is age-related macular degeneration.

21. The method according to claim 16, wherein the miRNA type exhibits a miRNA activity by binding to the region of the base sequence represented by SEQ ID NO: 1 of mRNA encoding VE-cadherin.

22. The method according to claim 17, wherein the miRNA type exhibits a miRNA activity by binding to the region of the base sequence represented by SEQ ID NO: 1 of mRNA encoding VE-cadherin.

23. The method according to claim 18, wherein the miRNA type exhibits a miRNA activity by binding to the region of the base sequence represented by SEQ ID NO: 1 of mRNA encoding VE-cadherin.

24. The method according to claim 16, wherein a portion constituting miRNA in a base sequence of the miRNA type includes the base sequence represented by SEQ ID NO: 2.

25. The method according to claim 17, wherein a portion constituting miRNA in a base sequence of the miRNA type includes the base sequence represented by SEQ ID NO: 2.

26. The method according to claim 18, wherein a portion constituting miRNA in a base sequence of the miRNA type includes the base sequence represented by SEQ ID NO: 2.

27. The method according to claim 16, wherein a portion constituting miRNA in the base sequence of the miRNA type includes a base sequence in which the following (A) or (B) binds to the 3′ terminal end of a base sequence represented by SEQ ID NO: 2:
(A) a base sequence represented by SEQ ID NO: 3; or
(B) a base sequence represented by SEQ ID NO: 3 in which one or a plurality of nucleotides is deleted, substituted, or added.

28. The method according to claim 17, wherein a portion constituting miRNA in the base sequence of the miRNA type includes a base sequence in which the following (A) or (B) binds to the 3′ terminal end of a base sequence represented by SEQ ID NO: 2:
(A) a base sequence represented by SEQ ID NO: 3; or
(B) a base sequence represented by SEQ ID NO: 3 in which one or a plurality of nucleotides is deleted, substituted, or added.

29. The method according to claim 18, wherein a portion constituting miRNA in the base sequence of the miRNA type includes a base sequence in which the following (A) or (B) binds to the 3′ terminal end of a base sequence represented by SEQ ID NO: 2:
(A) a base sequence represented by SEQ ID NO: 3; or
(B) a base sequence represented by SEQ ID NO: 3 in which one or a plurality of nucleotides is deleted, substituted, or added.

30. The method according to claim 16, wherein the number of bases of a portion constituting miRNA in the base sequence of the miRNA type is 19 to 25.

31. The method according to claim 17, wherein the number of bases of a portion constituting miRNA in the base sequence of the miRNA type is 19 to 25.

32. The method according to claim 18, wherein the number of bases of a portion constituting miRNA in the base sequence of the miRNA type is 19 to 25.


SEQUENCE LISTING (12 sequences)

SEQ ID NO: 1 - RNA, 9 nt, Homo sapiens
ucucaggga

SEQ ID NO: 2 - RNA, 9 nt, Homo sapiens
ucccugaga

SEQ ID NO: 3 - RNA, 13 nt, Homo sapiens
cccuaacuuguga

SEQ ID NO: 4 - DNA, 21 bp, Artificial
  primer
ccctgagaccctaacttgtga

SEQ ID NO: 5 - DNA, 20 bp, Artificial
  primer
cgcttcggcagcacatatac

SEQ ID NO: 6 - DNA, 21 bp, Artificial
  primer
aaaatatggaacgcttcacga

SEQ ID NO: 7 - DNA, 20 bp, Artificial
  primer
atcggttgttcaatgcgtcc

SEQ ID NO: 8 - DNA, 23 bp, Artificial
  primer
ccttcaggatttggtacatgaca

SEQ ID NO: 9 - DNA, 19 bp, Artificial
  primer
tcgttgcgctcttcgtgac

SEQ ID NO: 10 - DNA, 19 bp, Artificial
  primer
cagcccgcaaaacaggtag

SEQ ID NO: 11 - DNA, 19 bp, Artificial
  primer
gaaggtgaaggtcggagtc

SEQ ID NO: 12 - DNA, 20 bp, Artificial
  primer
gaagatggtgatgggatttc